(19) United States
(12) Patent Application Publication
(10) Pub. No.: US 2024/0108826 A1
(43) Pub. Date: Apr. 4, 2024

(54) INHALER SYSTEM

(71) Applicant: Norton (Waterford) Limited, Waterford (IE)

(72) Inventor: Mark Milton-Edwards, Cheshire (GB)

(73) Assignee: Norton (Waterford) Limited, Waterford (IE)

(21) Appl. No.: 18/558,126

(22) PCT Filed: Apr. 29, 2022
(86) PCT No.: PCT/EP2022/061579

§ 371 Date: Oct. 30, 2023

(30) Foreign Application Priority Data
Apr. 30, 2021 (GB) 2106254.2

(51) Int. Cl. A61M 15/00; A61K 31/573

(52) U.S. Cl. CPC A61M 15/0065; A61K 31/573; A61M 15/0026; A61M 2205/3334; A61M 2205/3553

(57) ABSTRACT

Provided is a pharmaceutical composition comprising an oral corticosteroid for use in post-exacerbation treatment of a respiratory disease in a subject, which post-exacerbation treatment includes an initial dose of the oral corticosteroid. The post-exacerbation treatment being continued until fulfilment of an inhalation parameter criterion by one or more post-exacerbation inhalations performed by the subject with at least one inhaler and/or fulfilment of a rescue inhaler usage criterion relating to post-exacerbation usage of a rescue inhaler configured to deliver a rescue medicament to the subject, at which point the dose of the oral corticosteroid is changed from the initial dose. Further provided are systems and methods for determining whether the inhalation parameter criterion is fulfilled and for comparing a post-exacerbation value of an inhalation parameter to a baseline value of the inhalation parameter.

### FIELD OF THE INVENTION

This disclosure relates to methods of treatment, systems and related methods for guiding the continued, or reduced use, of oral corticosteroids or other medicaments, such as biologics, for post-exacerbation treatment of a subject suffering from a respiratory disease, such as asthma and/or COPD.

### BACKGROUND OF THE INVENTION

Many respiratory diseases, such as asthma or chronic obstructive pulmonary disease (COPD), are life-long conditions where treatment involves the long-term administration of medicaments to manage the patients' symptoms and to decrease the risks of irreversible changes. There is currently no cure for diseases like asthma and COPD. Treatment takes two forms. First, a maintenance aspect of the treatment is intended to reduce airway inflammation and, consequently, control symptoms in the future. The maintenance therapy is typically provided by inhaled corticosteroids, alone or in combination with long-acting bronchodilators and/or muscarinic antagonists. Secondly, there is also a rescue (or reliever) aspect of the therapy, where patients are given rapid-acting bronchodilators to relieve acute episodes of wheezing, coughing, chest tightness and shortness of breath.

Patients suffering from a respiratory disease, such as asthma or COPD, may also experience episodic flare-ups, or exacerbations, in their respiratory disease, where symptoms rapidly worsen. In the worst case, exacerbations may be life-threatening.

Following clinical diagnosis of an exacerbation of a patient's respiratory disease, the patient may be treated with oral corticosteroids. Such administration of oral corticosteroids can also be combined with controlled flow oxygen, which requires hospitalization.

Post-exacerbation treatment comprising administration of oral corticosteroids can improve patient outcomes, and, where applicable, reduce the length of hospital stay. However, administration of oral corticosteroids is not without problem. In particular, various adverse drug reactions (ADRs) are associated with such use of oral corticosteroids.

### SUMMARY OF THE INVENTION

Accordingly, the present disclosure provides a pharmaceutical composition comprising an oral corticosteroid for use in post-exacerbation treatment of a respiratory disease in a subject, which post-exacerbation treatment includes an initial dose of the oral corticosteroid.

The present disclosure also provides systems and methods for guiding the continued, or reduced use, of oral corticosteroids.

Further provided is a method of post-exacerbation treatment of a respiratory disease in a subject, with the method comprising treating the subject with an initial dose of an oral corticosteroid.

The post-exacerbation treatment is continued until fulfilment of an inhalation parameter criterion by one or more post-exacerbation inhalations performed by the subject with at least one inhaler and/or fulfilment of a rescue inhaler usage criterion relating to post-exacerbation usage of a rescue inhaler configured to deliver a rescue medicament to the subject, at which point the dose of the oral corticosteroid is changed from the initial dose.

In spite of improved patient outcomes associated with administration of oral corticosteroids, the various adverse drug reactions (ADRs) associated with such use of oral corticosteroids are taken into account for determining, in particular, the duration of the treatment, and how the treatment should be stopped. Longer courses of oral corticosteroids tend to require the dose to be tapered towards the end of the treatment, known as weaning, rather than stopped abruptly.

It is desirable to monitor the subject's, in other words patient's, respiratory disease so that adjustment to the initial dose of the oral corticosteroid can be made accordingly. Such monitoring can, for example, be used to justify decreasing the dose relative to the initial dose should the patient's condition be observed to improve. For longer term oral corticosteroid use, such monitoring can guide a programmed reduction of oral corticosteroid dose.

Such monitoring can normally be challenging, particularly following discharge of the patient from hospital. Forced expiratory volume in the first second (FEV₁) can be used. However, such expiratory flow testing provides a burden for the patient in the form of an additional/different manoeuvre. This burden may lead to reduced amounts of data being gathered for justifying adjustment to the initial dose of the oral corticosteroid. Thus, the patient may remain on the initial dose of the oral corticosteroid for longer than is necessary or desirable, particularly in view of the risk of ADRs.

The post-exacerbation treatment according to the present disclosure is continued until fulfilment of an inhalation parameter criterion by one or more post-exacerbation inhalations performed by the subject with at least one inhaler and/or fulfilment of a rescue inhaler usage criterion relating to post-exacerbation usage of a rescue inhaler configured to deliver a rescue medicament to the subject. The post-exacerbation inhalations may be performed by the patient regardless of the aim of monitoring their respiratory disease. Thus, the monitoring can be implemented in a way which minimizes burden on the patient, and can thereby increase the likelihood that the requisite data is timely obtained to justify adjustment to the initial dose of the oral corticosteroid.

### BRIEF DESCRIPTION OF THE DRAWINGS

The present invention will now be described in more detail with reference to the accompanying drawings, which are not intended to be limiting:

FIG. 1 shows a block diagram of a system according to an embodiment;

FIG. 2 shows a system according to another embodiment;

FIG. 3A shows a flowchart of a method according to an embodiment;

FIG. 3B shows a flowchart of a method according to another embodiment;

FIG. 3C shows a flowchart of a method according to yet another embodiment;

FIG. 3D shows a flowchart of a method according to a further embodiment;

FIG. 4 shows a front perspective view of an inhaler;

FIG. 5 shows a cross-sectional interior perspective view of the inhaler shown in FIG. 4;

FIG. 6 provides an exploded perspective view of the example inhaler shown in FIG. 4;

FIG. 7 provides an exploded perspective view of a top cap and electronics module of the inhaler shown in FIG. 4; and

FIG. 8 shows a graph of airflow rate through the example inhaler shown in FIG. 4 versus pressure.

### DETAILED DESCRIPTION OF THE INVENTION

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the figures to indicate the same or similar parts.

Asthma and COPD are chronic inflammatory disease of the airways. They are both characterized by variable and recurring symptoms of airflow obstruction and bronchospasm. The symptoms include episodes of wheezing, coughing, chest tightness and shortness of breath.

The symptoms are managed by avoiding triggers and by the use of medicaments, particularly inhaled medicaments. The medicaments include inhaled corticosteroids (ICSs) and bronchodilators.

Inhaled corticosteroids (ICSs) are steroid hormones used in the long-term control of respiratory disorders. They function by reducing the airway inflammation. Examples include budesonide, beclomethasone (dipropionate), fluticasone (propionate or furoate), mometasone (furoate), ciclesonide and dexamethasone (sodium). Parentheses indicate preferred salt or ester forms. Particular mention should be made of budesonide, beclomethasone and fluticasone, especially budesonide, beclomethasone dipropionate, fluticasone propionate and fluticasone furoate.

Different classes of bronchodilators target different receptors in the airways. Two commonly used classes are β₂-agonists and anticholinergics.

β₂-Adrenergic agonists (or “β₂-agonists”) act upon the β₂-adrenoceptors which induces smooth muscle relaxation, resulting in dilation of the bronchial passages. They tend to be categorised by duration of action. Examples of long-acting β₂-agonists (LABAs) include formoterol (fumarate), salmeterol (xinafoate), indacaterol (maleate), bambuterol (hydrochloride), clenbuterol (hydrochloride), olodaterol (hydrochloride), carmoterol (hydrochloride), tulobuterol (hydrochloride) and vilanterol (triphenylacetate). Examples of short-acting β₂-agonists (SABA) are albuterol (sulfate) and terbutaline (sulfate). Particular mention should be made of formoterol, salmeterol, indacaterol and vilanterol, especially formoterol fumarate, salmeterol xinafoate, indacaterol maleate and vilanterol triphenylacetate.

Typically short-acting bronchodilators provide a rapid relief from acute bronchoconstriction (and are often called “rescue” or “reliever” medicines), whereas long-acting bronchodilators help control and prevent longer-term symptoms. However, some rapid-onset long-acting bronchodilators may be used as rescue medicines, such as formoterol (fumarate). Thus, a rescue medicine provides relief from acute bronchoconstriction. The rescue medicine is taken as-needed/prn (pro re nata). The rescue medicine may also be in the form of a combination product, e.g. ICS-formoterol (fumarate), typically budesonide-formoterol (fumarate). Thus, the rescue medicine is preferably a SABA or a rapid-acting LABA, more preferably albuterol (sulfate) or formoterol (fumarate), and most preferably albuterol (sulfate).

Albuterol (also known as salbutamol), typically administered as the sulfate salt, is a preferred rescue medicine of the present disclosure.

Anticholinergics (or “antimuscarinics”) block the neurotransmitter acetylcholine by selectively blocking its receptor in nerve cells. On topical application, anticholinergics act predominantly on the M₃ muscarinic receptors located in the airways to produce smooth muscle relaxation, thus producing a bronchodilatory effect. Examples of long-acting muscarinic antagonists (LAMAS) include tiotropium (bromide), oxitropium (bromide), aclidinium (bromide), ipratropium (bromide) glycopyrronium (bromide), oxybutynin (hydrochloride or hydrobromide), tolterodine (tartrate), trospium (chloride), solifenacin (succinate), fesoterodine (fumarate) and darifenacin (hydrobromide). Particular mention should be made of tiotropium, aclidinium, umeclidinium and glycopyrronium, especially tiotropium bromide, aclidinium bromide, umeclidinium bromide and glycopyrronium bromide.

A number of approaches have been taken in preparing and formulating these medicaments for delivery by inhalation, such as via a dry powder inhaler (DPI), a pressurized metered dose inhaler (pMDI) or a nebulizer.

According to the GINA (Global Initiative for Asthma) Guidelines, a step-wise approach is taken to the treatment of asthma. At step 1, which represents a mild form of asthma, the patient is given an as needed SABA, such as albuterol sulfate. The patient may also be given an as-needed low-dose ICS-formoterol, or a low-dose ICS whenever the SABA is taken. At step 2, a regular low-dose ICS is given alongside the SABA, or an as-needed low-dose ICS-formoterol. At step 3, a LABA is added. At step 4, the doses are increased and at step 5, further add-on treatments are included such as an anticholinergic or a low-dose oral corticosteroid. Thus, the respective steps may be regarded as treatment regimens, which regimens are each configured according to the degree of acute severity of the respiratory disease.

COPD is a leading cause of death worldwide. It is a heterogeneous long-term disease comprising chronic bronchitis, emphysema and also involving the small airways. The pathological changes occurring in patients with COPD are predominantly localised to the airways, lung parenchyma and pulmonary vasculature. Phenotypically, these changes reduce the healthy ability of the lungs to absorb and expel gases.

Bronchitis is characterised by long-term inflammation of the bronchi. Common symptoms may include wheezing, shortness of breath, cough and expectoration of sputum, all of which are highly uncomfortable and detrimental to the patient's quality of life. Emphysema is also related to long-term bronchial inflammation, wherein the inflammatory response results in a breakdown of lung tissue and progressive narrowing of the airways. In time, the lung tissue loses its natural elasticity and becomes enlarged. As such, the efficacy with which gases are exchanged is reduced and respired air is often trapped within the lung. This results in localised hypoxia, and reduces the volume of oxygen being delivered into the patient's bloodstream, per inhalation. Patients therefore experience shortness of breath and instances of breathing difficulty.

Patients living with COPD experience a variety, if not all, of these symptoms on a daily basis. Their severity will be determined by a range of factors but most commonly will be correlated to the progression of the disease. These symptoms, independent of their severity, are indicative of stable COPD and this disease state is maintained and managed through the administration of a variety drugs. The treatments are variable, but often include inhaled bronchodilators, anticholinergic agents, long-acting and short-acting β₂-agonists and corticosteroids. The medicaments are often administered as a single therapy or as combination treatments.

Patients are categorised by the severity of their COPD using categories defined in the GOLD Guidelines (Global Initiative for Chronic Obstructive Lung Disease, Inc.). The categories are labelled A-D and the recommended first choice of treatment varies by category. Patient group A are recommended a short-acting muscarinic antagonist (SAMA) prn or a short-acting β₂-aginist (SABA) prn. Patient group B are recommended a long-acting muscarinic antagonist (LAMA) or a long-acting β₂-aginist (LABA). Patient group C are recommended an inhaled corticosteroid (ICS)+a LABA, or a LAMA. Patient group D are recommended an ICS+a LABA and/or a LAMA.

Patients suffering from respiratory diseases like asthma or COPD suffer from periodic exacerbations beyond the baseline day-to-day variations in their condition. An exacerbation is an acute worsening of respiratory symptoms that require additional therapy, i.e. a therapy going beyond their maintenance therapy, including courses of oral steroid therapy.

For asthma, the additional therapy for a moderate exacerbation are repeated doses of SABA, oral corticosteroids and/or controlled flow oxygen (the latter of which requires hospitalization). A severe exacerbation adds an anticholinergic (typically ipratropium bromide), nebulized SABA or IV magnesium sulfate.

For COPD, the additional therapy for a moderate exacerbation are repeated doses of SABA, oral corticosteroids and/or antibiotics. A severe exacerbation adds controlled flow oxygen and/or respiratory support (both of which require hospitalization).

An exacerbation within the meaning of the present disclosure includes both moderate and severe exacerbations.

Provided is a pharmaceutical composition comprising an oral corticosteroid for use in post-exacerbation treatment of a respiratory disease in a subject, which post-exacerbation treatment includes an initial dose of the oral corticosteroid. By “oral corticosteroid” is meant a corticosteroid which is orally administered (and swallowed).

Further provided is a method of post-exacerbation treatment of a respiratory disease in a subject, the method comprising treating the subject with an initial dose of an oral corticosteroid.

The initial dose of the oral corticosteroid can be an initial daily dose of the oral corticosteroid. The initial daily dose can be administered at any suitable time of day, such as in the morning.

The pharmaceutical composition can be in tablet form, delayed-release tablet form, or in liquid form.

As an example of a liquid form, the oral corticosteroid is dissolved or dispersed in an oral solution. Such an oral solution can have any suitable concentration of the oral corticosteroid, such as 1 mg/mL to 20 mg/mL, preferably 5 mg/mL to 15 mg/mL.

The pharmaceutical composition can include any suitable oral corticosteroid, such as prednisone, prednisolone (sodium) and dexamethasone (sodium). Particular mention is made of prednisone and prednisolone (sodium). Prednisolone is the active metabolite of prednisone.

In some embodiments, the initial dose of prednisone or prednisolone is in the range of 5 to 60 mg daily for adults aged 18 years and older.

Preferably, the initial dose of prednisone or prednisolone is in the range of 25 to 60 mg daily for adults aged 18 years and older.

More preferably, the initial dose of prednisone or prednisolone is in the range of 30 to 50 mg daily for adults aged 18 years and older.

Most preferably, the initial dose of prednisone or prednisolone is in the range of 40 to 50 mg daily for adults aged 18 years and older.

In some embodiments, the initial dose of prednisone or prednisolone is in the range of 40 to 50 mg daily for children aged 12 to 17 years.

In some embodiments, the initial dose of prednisone or prednisolone is in the range of 1 to 2 mg/kg daily for children aged 1 month to 11 years.

The post-exacerbation treatment is continued until fulfilment of an inhalation parameter criterion (or in some non-limiting examples a plurality of inhalation parameter criteria) by one or more post-exacerbation inhalations performed by the subject with at least one inhaler, at which point the dose of the oral corticosteroid is changed from the initial dose.

Alternatively or additionally, the post-exacerbation treatment is continued until fulfilment of a rescue inhaler usage criterion relating to post-exacerbation usage of a rescue inhaler configured to deliver a rescue medicament to the subject (or in some non-limiting examples a plurality of rescue inhaler usage criteria).

Post-exacerbation treatment comprising administration of oral corticosteroids can improve patient outcomes, and potentially reduce the length of hospital stay. However, administration of oral corticosteroids is not without problem. In particular, various adverse drug reactions (ADRs) are associated with such use of oral corticosteroids.

ADRs are, in particular, taken into account for determining the duration of the treatment, and how the treatment should be stopped. Longer courses of oral corticosteroids tend to require the dose to be tapered towards the end of the treatment, known as weaning, rather than stopped abruptly. But this may be done with little or no empirical data to support the reduction.

It is desirable to monitor the patient's respiratory disease so that adjustment to the initial dose of the oral corticosteroid can be made accordingly. Such monitoring can, for example, be used to justify decreasing of the dose relative to the initial dose should the patient's condition be observed to improve. For longer term oral corticosteroid use, such monitoring can guide a programmed reduction, or increase of oral corticosteroid dose.

The post-exacerbation treatment according to the present disclosure is continued until fulfilment of an inhalation parameter criterion by one or more post-exacerbation inhalations performed by the subject with at least one inhaler and/or until fulfilment of a rescue inhaler usage criterion relating to post-exacerbation usage of a rescue inhaler configured to deliver a rescue medicament to the subject. The post-exacerbation inhalations may be performed by the patient regardless of the aim of monitoring their respiratory disease. Thus, the monitoring can be implemented in a way which minimizes burden on the patient, and can thereby increase the likelihood that the requisite data is timely obtained to justify adjustment to the initial dose of the oral corticosteroid.

In certain embodiments, the dose of the oral corticosteroid is lowered relative to the initial dose following fulfilment of the inhalation parameter criterion and/or the rescue inhaler usage criterion. Fulfilment of either or both of these criteria may thus indicate that the patient has recovered to the extent that lowering the dose of the oral corticosteroid is justified. In this way, fulfilment of either or both of these criteria can assist to balance the risk associated with the patient's respiratory disease with the risks posed by the ADRs associated with the oral corticosteroids.

In some embodiments, the dose of oral corticosteroid is lowered to zero following fulfilment of the inhalation parameter criterion and/or the rescue inhaler usage criterion. In this case, weaning off the oral corticosteroid may not be justified due to the oral corticosteroid having been administered to the patient, e.g. daily, over a short period, such as less than two weeks. The inhalation parameter criterion and/or the rescue inhaler usage criterion nonetheless provides a guide as to when the administration of the oral corticosteroid can be safely discontinued.

In alternative embodiments, the initial dose is first lowered to a subsequent dose based on fulfilment of the inhalation parameter criterion and/or the rescue inhaler usage criterion, and the subsequent dose is then lowered to a further subsequent dose which is lower than the subsequent dose based on fulfilment of a further inhalation parameter criterion and/or a further rescue inhaler usage criterion.

In this manner, the patient can be progressively weaned off the oral corticosteroid based on monitoring their condition via the inhalations performed with their inhaler(s).

Such inhalation parameter- and/or rescue inhaler usage-guided weaning may be of particular use when the oral corticosteroid has been administered to the patient, e.g. daily, over a relatively prolonged period, such as for two weeks or more. Such a relatively prolonged period of oral corticosteroid administration can, for example, be used due to severity of the respiratory disease and/or concerns relating to adrenal insufficiency.

Alternatively or additionally, the inhalation parameter criterion and/or the rescue inhaler usage criterion may provide a way of justifying an increase of the dose of the oral corticosteroid relative to the initial dose. For example, the dose of the oral corticosteroid may be increased from the initial dose following failure to fulfil the inhalation parameter criterion and/or the rescue inhaler usage criterion within a predetermined time period, such as within 1 to 3 weeks.

Each of the at least one inhaler may comprise a sensor system configured to measure a post-exacerbation value of an inhalation parameter.

The rescue inhaler may comprise a use determination system configured to determine post-exacerbation usage of the rescue inhaler.

More generally, fulfilment of the inhalation parameter criterion and/or the rescue inhaler usage criterion may be established empirically and/or based on previous pre-exacerbation individual baseline values, as will be described in more detail herein.

In at least some embodiments, the at least one inhaler comprises a maintenance inhaler configured to deliver a maintenance medicament to the subject during the one or more post-exacerbation inhalations.

The patient may thus begin or resume maintenance therapy at the same time as the post-exacerbation treatment with the oral corticosteroid. Since the patient routinely performs inhalations in order to administer the maintenance medicament, the monitoring of their condition may not require additional measurements, such as spirometry measurements for determining expiratory flow parameters, to be performed by the patient. This alleviates burden on the patient, as previously described.

In a non-limiting example, the maintenance medicament selected from budesonide, beclomethasone (dipropionate), fluticasone (propionate or furoate), and salmeterol (xinafoate) combined with fluticasone (propionate or furoate).

Alternatively or additionally, the at least one inhaler comprises a rescue inhaler configured to deliver a rescue medicament to the subject during the one or more post-exacerbation inhalations.

The rescue inhaler included in the at least one inhaler may be the same as or different from the rescue inhaler whose post-exacerbation usage by the subject is used to determine fulfilment of the rescue inhaler usage criterion (in embodiments in which the rescue inhaler usage criterion is utilized).

Thus, the rescue inhalations performed by the patient can be used to monitor their condition with a view to appropriately adjusting the dose of the oral corticosteroid from the initial dose. Burden on the patient can be alleviated because rescue inhaler usage data and/or inhalation parameter data gathered during necessary rescue inhalations may obviate the requirement for additional spirometry measurements.

Since the rescue medicament may be used as-needed by the subject, usage of the rescue inhaler may provide a useful indicator to track post-exacerbation recovery. Fewer rescue inhalations, e.g. fewer daily rescue inhalations, may, for example, point to an improvement in the subject's condition.

In certain embodiments, the subject has a baseline usage of the rescue inhaler, and fulfilment of the rescue inhaler usage criterion is determined based on a comparison of a post-exacerbation usage of the rescue inhaler obtained from the more or more post-exacerbation inhalations to the baseline rescue inhaler usage.

The baseline usage of the rescue inhaler may, for example, be determined when the subject is not experiencing an exacerbation, and is neither in a period of worsening prior to such an exacerbation, nor in a period of post-exacerbation recovery following an exacerbation.

The baseline usage of the rescue inhaler may, for example, be determined from rescue inhaler usage in a baseline period. Such a baseline period may, for instance, have a duration of 5 to 30 days.

The rescue inhaler usage criterion may, for example, be fulfilled by the number of daily post-exacerbation rescue inhaler uses being equal to or lower than a given threshold daily number of rescue inhaler uses, e.g. for a minimum period of time, such as a minimum number of days. The given threshold may, for instance, be defined by a baseline daily number of rescue inhaler uses, such as a baseline average, e.g. mean, daily number of rescue inhaler uses performed by the subject.

Such a baseline daily number of rescue inhaler uses, for example a baseline average, e.g. mean, daily number of rescue inhaler uses, may be determined when the subject is not experiencing an exacerbation, and is neither in a period of worsening prior to such an exacerbation, nor in a period of post-exacerbation recovery following an exacerbation.

The rescue medicament is as defined hereinabove and is typically a SABA, a SAMA, a rapid-onset LABA, such as formoterol (fumarate), a combination of a SAMA with LABA and/or an ICS, or a combination of a LAMA with LABA and/or an ICS.

The rescue medicament in the form of a combination product may be, for example, ICS-formoterol (fumarate), typically budesonide-formoterol (fumarate). Such an approach is termed “MART” (maintenance and rescue therapy).

In a non-limiting example, the rescue medicament is selected from albuterol (sulfate), formoterol (fumarate), budesonide combined with formoterol (fumarate), beclomethasone (dipropionate) combined with albuterol (sulfate), and fluticasone (propionate or furoate) combined with albuterol (sulfate).

In some embodiments, determination of fulfilment of the inhalation parameter criterion is based on an inhalation parameter relating to airflow through the inhaler during the one or more post-exacerbation inhalations performed by the subject with the at least one inhaler.

In some embodiments, the subject has a baseline lung function indicated by a baseline value of an inhalation parameter, and fulfilment of the inhalation parameter criterion is determined based on a comparison of a post-exacerbation value of the inhalation parameter obtained from said one or more post-exacerbation inhalations to the baseline value.

A sensor system included in the at least one inhaler may be configured to measure the post-exacerbation value of the inhalation parameter.

The baseline value of the inhalation parameter can, for example, be established by measurement of one or more non-exacerbation values of the inhalation parameter during a period in which the patient is not experiencing an exacerbation.

The baseline value may, for example, be determined from inhalation(s) performed by the subject during a baseline period. Such a baseline period may, for instance, have a duration of 5 to 30 days.

Such one or more non-exacerbation values may also, for example, be measured by the sensor system included in the at least one inhaler. Alternatively, the one or more non-exacerbation values of the inhalation parameter may be calculated from a spirometer, in other words expiratory flow, measurements taken when the patient is not exacerbating. Such a calculation may be based on a correlation established between, for instance, peak expiratory flow and peak inhalation flow.

The inhalation parameter criterion may be, more generally, regarded as a requirement relating to airflow during the one or more post-exacerbation inhalations using the at least one inhaler.

The inhalation parameter can be regarded as a parameter relating to airflow during the inhalation performed by the subject using the at least one inhaler. The inhalation parameter may act as a proxy for the lung condition of the subject.

Any suitable inhalation parameter can be considered. In a non-limiting example, the inhalation parameter comprises, or in some cases consists of, one or more of a peak inhalation flow, an inhalation volume, an inhalation duration, and a time taken to reach peak inhalation flow, also referred to herein as a “time to peak inhalation flow”.

Increasing peak inhalation flow, greater inhalation volumes, more prolonged inhalation durations, and/or shorter times to reach peak inhalation flow during post-exacerbation inhalations performed by the patient using the inhaler(s) can point to improvement in the patient's lung condition. Conversely, these parameters staying the same or declining in the case of the peak inhalation flow, inhalation volume and inhalation duration (and increasing in the case of the time to reach peak inhalation flow) can point to the patient's lung condition worsening. Such information can therefore be valuable in determining if, when and/or how to change the initial dose of the oral corticosteroid.

More generally, an assessment of improvement or worsening in the condition of the patient can be provided via the comparison between the post-exacerbation value and the baseline value. Such a comparison indicating reversion towards or to the baseline value, or even an improvement beyond the baseline value, can be used to justify changing, and in particular lowering, of the dose of the oral corticosteroid with respect to the initial dose.

Such a comparison can be quantified in any suitable manner. In a non-limiting example, the inhalation parameter criterion is fulfilled by the post-exacerbation value being within a predetermined tolerance defined for the baseline value.

This may be regarded as an assessment of “proximity” of the post-exacerbation value to baseline or pre-exacerbation/non-exacerbation levels.

In certain non-limiting examples, the post-exacerbation value of the inhalation parameter increases towards the baseline value during reversion to said baseline lung function. This is the case when, for example, the inhalation parameter is a peak inhalation flow, an inhalation volume, and/or an inhalation duration.

In such examples in which the inhalation parameter increases towards the baseline value during reversion to said baseline lung function, the inhalation parameter criterion can be fulfilled by the post-exacerbation value being at least 70% of the baseline value, at least 80% of the baseline value, or at least 90% of the baseline value.

In at least some embodiments, the post-exacerbation value comprises an average of a plurality of post-exacerbation values of the inhalation parameter obtained from a plurality of said post-exacerbation inhalations.

By basing fulfilment of the inhalation parameter criterion based on a plurality of post-exacerbation values, e.g. gathered over a plurality of days, the dose of the oral corticosteroid from the initial dose may be more reliably changed from the initial dose. In other words, the confidence associated with changing the dose of the oral corticosteroid from the initial dose can increase by using more than one inhalation parameter data points, particular over a number of post-exacerbation days. An apposite number of post-exacerbation values can be used to determine the average considering the variability of such values.

Also provided is a method, e.g. a computer implemented method, comprising receiving a baseline value of an inhalation parameter indicative of a baseline lung function of the subject, and receiving a post-exacerbation value of the inhalation parameter determined from one or more post-exacerbation inhalations using at least one inhaler.

The method further comprises determining, based on a comparison between the post-exacerbation value and the baseline value, fulfilment of an inhalation parameter criterion for, e.g. suitable for, changing the dose of an oral corticosteroid being administered to the subject post-exacerbation from an initial dose.

Alternatively or additionally, a method comprises receiving a baseline rescue inhaler usage, e.g. a baseline number of daily rescue inhalations; and receiving a post-exacerbation rescue inhaler usage, e.g. a number of daily post-exacerbation rescue inhalations. Such a method further comprises determining, based on a comparison between the post-exacerbation rescue inhaler usage and the baseline rescue inhaler usage, fulfilment of a rescue inhaler usage criterion for, e.g. suitable for, changing the dose of an oral corticosteroid being administered to the subject post-exacerbation from an initial dose.

The rescue inhaler usage criterion may, for example, be fulfilled by the number of daily post-exacerbation rescue inhaler uses being equal to or lower than a given threshold daily number of rescue inhaler uses. The given threshold may, for instance, be defined by a baseline daily number of rescue inhaler uses, such as a baseline average, e.g. mean, daily number of rescue inhaler uses performed by the subject, as previously described.

The method(s) may thus provide automated determination of when the inhalation parameter criterion and/or the rescue inhaler usage criterion is or are fulfilled such that the dose of the oral corticosteroid can be changed from the initial dose.

In some embodiments, the inhalation parameter comprises, or in some cases consists of, one or more of a peak inhalation flow, an inhalation volume, an inhalation duration, and a time taken to reach peak inhalation flow, as previously described.

Alternatively or additionally, the inhalation parameter criterion is fulfilled by the post-exacerbation value being within a predetermined tolerance defined for the baseline value. This can provide an assessment of “proximity” of the post-exacerbation value to baseline or pre-exacerbation/non-exacerbation levels.

In some embodiments, the post-exacerbation value of the inhalation parameter increases towards the baseline value during reversion to said baseline lung function. This is the case when, for example, the inhalation parameter is a peak inhalation flow, an inhalation volume, and/or an inhalation duration.

In such embodiments, the inhalation parameter criterion can be fulfilled by the post-exacerbation value being at least 70% of the baseline value, at least 80% of the baseline value, or at least 90% of the baseline value.

In at least some embodiments, the post-exacerbation value comprises an average of a plurality of post-exacerbation values of the inhalation parameter obtained from a plurality of said post-exacerbation inhalations.

An apposite number of post-exacerbation values can be used to determine the average considering the variability of such values, as previously explained.

Further provided is a method comprising receiving a baseline value of an inhalation parameter indicative of a baseline lung function of a subject, and receiving a post-exacerbation value of the inhalation parameter determined from one or more post-exacerbation inhalations performed by the subject using at least one inhaler. The method further comprises controlling a user interface to communicate the baseline value, and controlling the user interface to communicate the post-exacerbation value such as to permit comparison between the post-exacerbation value and the at least one baseline value.

Alternatively or additionally, a method comprises receiving a baseline rescue inhaler usage, e.g. a baseline number of daily rescue inhaler uses; and receiving a post-exacerbation rescue inhaler usage, e.g. a number of daily post-exacerbation rescue inhalations. This method further comprises controlling a user interface to communicate the baseline rescue inhaler usage; and controlling the user interface to communicate the post-exacerbation rescue inhaler usage such as to permit comparison between the post-exacerbation rescue inhaler usage and the baseline rescue inhaler usage.

By controlling the user interface such as to permit comparison between the baseline value and the post-exacerbation value and/or comparison between the baseline rescue inhaler usage and the post-exacerbation rescue inhaler usage, the method assists the user, e.g. a clinician, in the technical task of determining whether and/or how to change the dose of the oral corticosteroid from the initial dose.

Such communication may be implemented in any suitable manner, for example by simultaneous display of the baseline value and post-exacerbation value and/or simultaneous display of the baseline rescue inhaler usage and the post-exacerbation rescue inhaler usage on a suitable display device. Such simultaneous display can take any suitable form provided that the comparison can be made.

Particular mention is made of graphical display of the values, which may assist the healthcare provider to discern any change in the post-exacerbation value and/or post-exacerbation rescue inhaler usage over time as well as compare a current post-exacerbation value with the baseline value and/or compare post-exacerbation rescue inhaler usage with the baseline rescue inhaler usage. The baseline value and/or the baseline rescue inhaler usage may, for instance, appear as a line superimposed onto a graph of the post-exacerbation value and/or post-exacerbation rescue inhaler usage (respectively) versus time.

Alternative ways of communicating the baseline value and the post-exacerbation value and/or the baseline rescue inhaler usage and the post-exacerbation rescue inhaler usage such as to permit comparison between them will also be apparent to the skilled person. For example, the baseline value/baseline rescue inhaler usage may be marked on a graduated (e.g. graphical) dial and change of the post-exacerbation value/post-exacerbation rescue inhaler usage may be represented by movement of a needle relative to the dial.

It should be understood that the method(s) summarized above comprising determining fulfilment of the inhalation parameter criterion and/or fulfilment of the rescue inhaler usage criterion may be combined with the method(s) enabling comparison between the baseline value and the post-exacerbation value and/or comparison between the baseline rescue inhaler usage and the post-exacerbation rescue inhaler usage. Thus, method(s) is or are provided combining these functions.

Irrespective of whether the method comprises determining fulfilment of the inhalation parameter criterion or enabling comparison between the baseline value and the post-exacerbation value, the baseline value can be determined from one or more inhalations using the at least one inhaler when the subject is not experiencing an exacerbation, and is neither in a period of worsening prior to such an exacerbation, nor in a period of post-exacerbation recovery following an exacerbation.

Moreover, irrespective of whether the method comprises determining fulfilment of the rescue inhaler usage criterion or enabling comparison between the baseline rescue inhaler usage and the post-exacerbation rescue inhaler usage, the baseline rescue inhaler usage can be when the subject is not experiencing an exacerbation, and is neither in a period of worsening prior to such an exacerbation, nor in a period of post-exacerbation recovery following an exacerbation, as previously described.

The present disclosure also provides a computer program comprising computer program code which is configured, when said program is run on one or more physical computing devices, to cause said one or more physical computing devices to implement one or more of the above-described methods.

Similarly, the present disclosure provides one or more non-transitory computer readable media having a computer program stored thereon, the computer program comprising computer program code which is configured, when said computer program is run on one or more physical computing devices, to cause said one or more physical computing devices to implement one or more of the above-described methods.

A system is provided comprising at least one inhaler having a sensor system configured to determine a value of an inhalation parameter from an inhalation performed by a subject using the at least one inhaler.

The system further comprises one or more processors configured to receive a baseline value of an inhalation parameter indicative of a baseline lung function of the subject, and receive a post-exacerbation value of the inhalation parameter determined from one or more post-exacerbation inhalations using the at least one inhaler. The one or more processors is or are further configured to determine, based on a comparison between the post-exacerbation value and the baseline value, fulfilment of an inhalation parameter criterion for, e.g. suitable for, changing the dose of an oral corticosteroid being administered to the subject post-exacerbation from an initial dose.

Alternatively or additionally, a system is provided, which system comprises: a rescue inhaler having a use determination system configured to determine usage of the rescue inhaler by a subject in order to deliver a rescue medicament to the subject; and one or more processors configured to: receive a baseline rescue inhaler usage; receive a post-exacerbation rescue inhaler usage; and determine, based on a comparison between the post-exacerbation rescue inhaler usage and the baseline rescue inhaler usage, fulfilment of a rescue inhaler usage criterion for changing the dose of an oral corticosteroid being administered to the subject post-exacerbation from an initial dose.

The system(s) may thus provide automated determination of when the inhalation parameter criterion and/or the rescue inhaler usage criterion is or are fulfilled such that the dose of the oral corticosteroid can be changed from the initial dose.

The system may, for example, include a user interface configured to provide an output responsive to the determination of fulfilment of the inhalation parameter criterion and/or the rescue inhaler usage criterion.

Such an output may, for instance, be in the form of a notification to a healthcare provider. Such a notification may, for example, include an indication of if, when and/or how the dose of the oral corticosteroid should be changed from the initial dose.

Alternatively or additionally, the system may comprise a medication container in communication, for example wireless communication, with the one or more processors. The medication container contains the oral corticosteroid, e.g. in tablet or delayed release tablet form. In such a non-limiting example, the medication container may be responsive to the determination that the inhalation parameter criterion and/or the rescue inhaler usage criterion is fulfilled such as to assist the subject to change the dose of the oral corticosteroid from the initial dose.

As an alternative or in addition to the functionality of the system described above, the present disclosure provides a system comprising at least one inhaler having a sensor system configured to determine a value of an inhalation parameter from an inhalation performed by a subject using the at least one inhaler, and a user interface.

The system further comprises one or more processors configured to receive a baseline value of an inhalation parameter indicative of a baseline lung function of a subject, and receive a post-exacerbation value of the inhalation parameter determined from one or more post-exacerbation inhalations performed by the subject using at least one inhaler. The one or more processors is or are further configured to control the user interface to communicate the baseline value, and control the user interface to communicate the post-exacerbation value such as to permit comparison between the post-exacerbation value and the baseline value.

Alternatively or additionally, a system is provided, which system comprises a rescue inhaler having a use determination system configured to determine usage of the rescue inhaler by a subject in order to deliver a rescue medicament to the subject; and one or more processors configured to: receive a baseline rescue inhaler usage; receive a post-exacerbation rescue inhaler usage; control a user interface to communicate the baseline rescue inhaler usage; and control the user interface to communicate the post-exacerbation rescue inhaler usage such as to permit comparison between the post-exacerbation rescue inhaler usage and the baseline rescue inhaler usage.

By the user interface being controlled to communicate the baseline value and the post-exacerbation value such as to permit comparison between the baseline value and the post-exacerbation value and/or communicate the baseline rescue inhaler usage and the post-exacerbation rescue inhaler usage such as to permit comparison between the post-exacerbation rescue inhaler usage and the baseline rescue inhaler usage, the system assists the user, e.g. a clinician, in the technical task of determining whether and/or how to change the dose of the oral corticosteroid from the initial dose.

More generally, any embodiments discussed in respect of the method of post-exacerbation treatment and pharmaceutical composition for the above-described use may be applied to the methods, computer program, and non-transitory computer readable media, and systems, and any embodiments described in respect of the methods, computer program, and non-transitory computer readable media, and systems may be applied to the method of post-exacerbation treatment and pharmaceutical composition.

Whilst the above description has focused on post-exacerbation treatment comprising administration of an oral corticosteroid, the principles are more broadly applicable. The present disclosure accordingly provides a pharmaceutical composition comprising a medicament for use in post-exacerbation treatment of a respiratory disease in a subject, which post-exacerbation treatment includes an initial dose of the medicament, said post-exacerbation treatment being continued until fulfilment of an inhalation parameter criterion by one or more post-exacerbation inhalations performed by the subject with at least one inhaler and/or fulfilment of a rescue inhaler usage criterion relating to post-exacerbation usage of a rescue inhaler configured to deliver a rescue medicament to the subject, at which point the dose of the medicament is changed from the initial dose.

The medicament can be or comprise any drug employed in the treatment of a respiratory disease, e.g. in post-exacerbation treatment. For example, the medicament may be an ICS, LABA, LAMA, SABA, SAMA or any combination of such medicaments. Specific examples of such ICS, LABA, LAMA, SABA, SAMA medicaments have been identified herein above.

The medicament can be, for example, a biologics medication for treatment of the subject's respiratory disease, e.g. for post-exacerbation treatment of the respiratory disease. The biologics medication may, for example, be or comprise one or more of omalizumab, mepolizumab, reslizumab, benralizumab and dupilumab.

The term “biologics medication” may refer to a medicine that contains one or more active substances made by or derived from a biological source.

The relatively high cost of biologics medications means that a course of biologics medication tends to require careful consideration and justification. The systems and methods according to the present disclosure may provide a reliable metric to justify if, when and/or how to change from an initial dose of a biologics medication. For example, should the inhalation parameter criterion and/or the rescue inhaler usage criterion be fulfilled, lowering the dose of biologics medication may be justified.

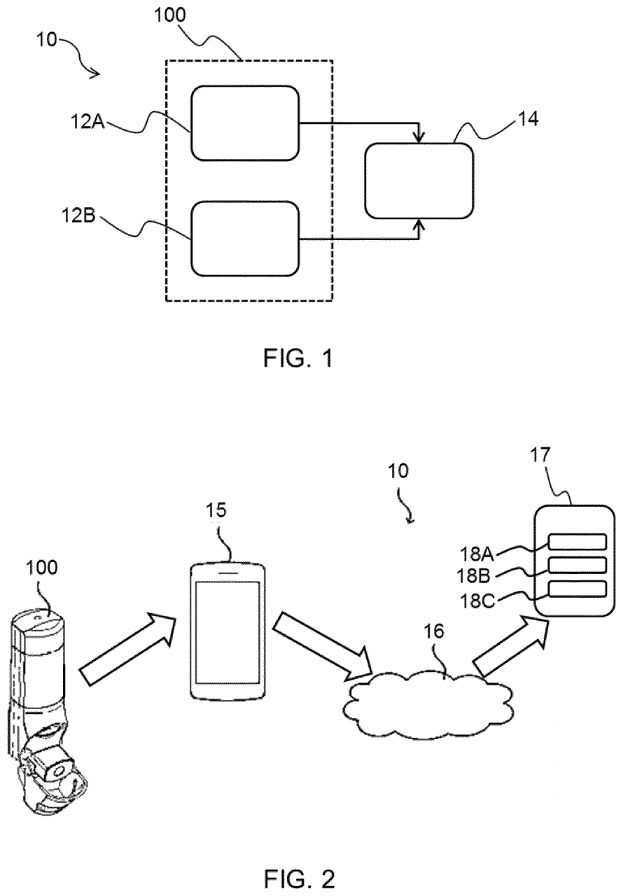

FIG. 1 shows a block diagram of a system 10 according to an embodiment. The system 10 comprises a first inhaler 100 and one or more processors 14. The first inhaler 100 may be used to deliver a maintenance medicament or a rescue medicament, such as a SABA, to the subject. The SABA may include, for example, albuterol. The first inhaler 100 may include a sensor system 12A, and/or a use determination system 12B.

The system 10 may, for example, be alternatively termed “an inhaler assembly”.

A sensor system 12A may be configured to measure a value of the inhalation parameter from an inhalation performed by a subject using the first inhaler. The sensor system 12A may, for example, comprise one or more sensors, such as one or more pressure sensors, temperature sensors, humidity sensors, orientation sensors, acoustic sensors, and/or optical sensors. The pressure sensor(s) may include a barometric pressure sensor (e.g. an atmospheric pressure sensor), a differential pressure sensor, an absolute pressure sensor, and/or the like. The sensors may employ microelectromechanical systems (MEMS) and/or nanoelectromechanical systems (NEMS) technology.

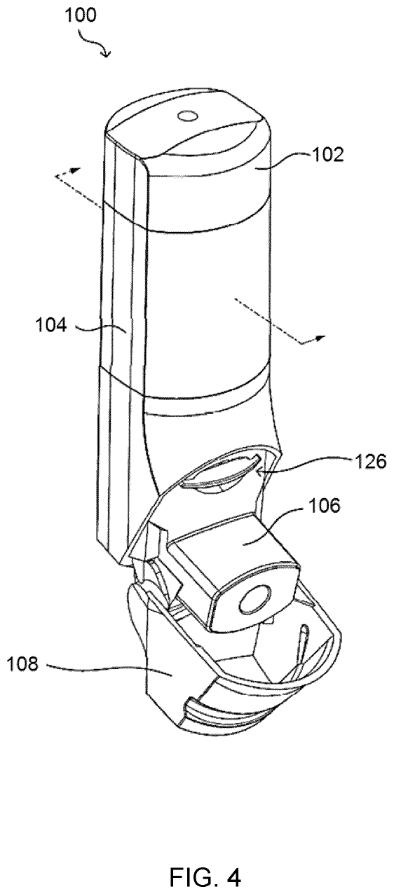

A pressure sensor(s) may be particularly suitable for measuring the parameter, since the airflow during inhalation by the subject may be monitored by measuring the associated pressure changes. As will be explained in greater detail with reference to FIGS. 4-8, a pressure sensor may be, for instance, located within or placed in fluid communication with a flow pathway through which air and the medicament is drawn by the subject during inhalation. Alternative ways of measuring the parameter, such as via a suitable flow sensor, will also be apparent to the skilled person.

Alternatively or additionally, the sensor system 12A may comprise a differential pressure sensor. The differential pressure sensor may, for instance, comprise a dual port type sensor for measuring a pressure difference across a section of the air passage through which the subject inhales. A single port gauge type sensor may alternatively be used. The latter operates by measuring the difference in pressure in the air passage during inhalation and when there is no flow. The difference in the readings corresponds to the pressure drop associated with inhalation.

Whilst not shown in FIG. 1, the system 10 may further comprise a second inhaler for delivering a medicament, such as a rescue or maintenance medicament to the subject. The first and second inhalers may, for example, be configured to deliver different medicaments from each other. The second inhaler may include a sensor system 12A and/or a use determination system 12B that is distinct from the sensor system 12A and the use determination system 12B of the first inhaler 100. The sensor system 12A of the second inhaler may be configured to measure the value of the inhalation parameter from an inhalation performed by a subject using the second inhaler. For example, the sensor system 12A may include a further pressure sensor, such as a further microelectromechanical system pressure sensor or a further nanoelectromechanical system pressure sensor, in order to measure the value of the inhalation parameter.

In this manner, inhalation of medicaments, e.g. rescue and/or maintenance medicaments, may be used to gather information relating to the subject's lung function and/or lung health.

Each inhalation may be associated with a decrease in the pressure in the airflow channel relative to when no inhalation is taking place. The point at which the pressure is at its lowest may correspond to the peak inhalation flow. The sensor system 12A may detect this point in the inhalation. The peak inhalation flow may vary from inhalation to inhalation, and may depend on the clinical condition of the subject. An increasing peak inhalation flow may point to the patient's condition improving following the exacerbation.

The pressure change associated with each inhalation may alternatively or additionally be used to determine an inhalation volume. This may be achieved by, for example, using the pressure change during the inhalation measured by the sensor system 12A to first determine the flow rate over the time of the inhalation, from which the total inhaled volume may be derived. Inhalations having larger volumes may indicate improvement in the patient's condition following the exacerbation.

The pressure change associated with each inhalation may alternatively or additionally be used to determine an inhalation duration. The time may be recorded, for example, from the first decrease in pressure measured by the pressure sensor 12A, coinciding with the start of the inhalation, to the pressure returning to a pressure corresponding to no inhalation taking place. Longer inhalation durations may point to patient recovery since the subject's capacity for inhaling for longer may be increased as the post-exacerbation treatment progresses.

In an embodiment, the parameter includes the time to peak inhalation flow, e.g. as an alternative or in addition to the peak inhalation flow, the inhalation volume and/or the inhalation duration. This time to peak inhalation flow parameter may be recorded, for example, from the first decrease in pressure measured by the sensor system 12A, coinciding with the start of the inhalation, to the pressure reaching a minimum value corresponding to peak flow. A patient whose condition is improving may tend to take less time to achieve peak inhalation flow. Note that this is an example of an inhalation parameter whose value may increase during an exacerbation. Subsequently, its post-exacerbation value may revert towards the baseline value by decreasing. In this case, an inhalation parameter criterion may be fulfilled when the post-exacerbation value is less than a predetermined threshold, which may be defined in proportion to the baseline value. For example, the inhalation parameter criterion may be fulfilled when the post-exacerbation value is less than 130%, or less than 120%, or less than 110% of the baseline value.

In a non-limiting example, the first and/or second inhalers may be configured such that, for a normal inhalation, the respective medicament is dispensed during approximately 0.5 s following the start of the inhalation. A subject's inhalation only reaching peak inhalation flow after the 0.5 s has elapsed, such as after approximately 1.5 s, may be partially indicative of the subject's lung condition being impaired. Improvement in the subject's condition may accordingly be observed via shorter times, in particular approaching the specified about 0.5 s time, to reach peak inhalation flow.

In the non-limiting example shown in FIG. 1, the use determination system 12B is configured to register inhalation(s) performed by the subject (e.g. each rescue inhalation by the subject when the inhaler is a rescue inhaler, or each maintenance inhalation by the subject when the inhaler is a maintenance inhaler).

In a non-limiting example, the first inhaler 100 may comprise a medicament reservoir (not shown in FIG. 1), and a dose metering assembly (not shown in FIG. 1) configured to meter a dose of the rescue medicament from the reservoir. The use determination system 12B may be configured to register the metering of the dose by the dose metering assembly, each metering being thereby indicative of the rescue inhalation performed by the subject using the first inhaler 100. Accordingly, the inhaler 100 may be configured to monitor the number of rescue inhalations of the medicament, since the dose must be metered via the dose metering assembly before being inhaled by the subject. One non-limiting example of the metering arrangement will be explained in greater detail with reference to FIGS. 4-8.

Alternatively or additionally, the use determination system 12B may register each inhalation in different manners and/or based on additional or alternative feedback that are apparent to the skilled person. For example, the use determination system 12B may be configured to register an inhalation by the subject when the feedback from the sensor system 12A indicates that an inhalation by the user has occurred (e.g. when a pressure measurement or flow rate exceeds a predefined threshold associated with a successful inhalation). Further, in some examples, the use determination system 12B may be configured to register an inhalation when a switch of the inhaler or a user input of an external device (e.g. touchscreen of a smartphone) is manually actuated by the subject prior to, during or after inhalation.

A sensor (e.g. a pressure sensor) may, for example, be included in the use determination system 12B in order to register each inhalation. In such an example, the use determination system 12B and the sensor system 12A may employ respective sensors (e.g. pressure sensors), or a common sensor (e.g. a common pressure sensor) which is configured to fulfil both use-detecting and inhalation parameter sensing functions.

When a sensor is included in the use determination system 12B, the sensor may, for instance, be used to confirm that, or assess the degree to which, a dose metered via the dose metering assembly is inhaled by the user, as will be described in greater detail with reference to FIGS. 4-8.

In an embodiment, the sensor system 12A and/or the use determination system 12B includes an acoustic sensor. The acoustic sensor in this embodiment is configured to sense a noise generated when the subject inhales through the respective inhaler. The acoustic sensor may include, for example, a microphone.

In a non-limiting example, the respective inhaler may comprise a capsule which is arranged to spin when the subject inhales though the device; the spinning of the capsule generating the noise for detection by the acoustic sensor. The spinning of the capsule may thus provide a suitably interpretable noise, e.g. rattle, for deriving use and/or inhalation parameter data.

An algorithm may, for example, be used to interpret the acoustic data in order to determine use data (when the acoustic sensor is included in the use determination system 12B) and/or the value for the inhalation parameter relating to airflow during the inhalation (when the acoustic sensor is included in the sensor system 12A).

For instance, an algorithm as described by P. Colthorpe et al., “Adding Electronics to the Breezhaler®: Satisfying the Needs of Patients and Regulators”, Respiratory Drug Delivery 2018, 1, 71-80 may be used. Once the generated sound is detected, the algorithm may process the raw acoustic data to generate the use and/or inhalation parameter data.

The one or more processors 14 included in the system 10 can be configured in various ways. As schematically shown in FIG. 1 by the arrows between the sensor system 12A and the processor 14, the processor 14 may receive the inhalation parameter data from the sensor system 12A. In a similar way, the one or more processors 14 can receive usage data from the use determination system 12B.

Fulfilment of the rescue inhaler usage criterion is based on post-exacerbation usage of the rescue inhaler. Since the rescue medicament may be used as-needed by the subject, usage of the rescue inhaler may provide a useful indicator to track post-exacerbation recovery. Fewer post-exacerbation rescue inhalations, e.g. fewer daily rescue inhalations, as determined via the use determination system 12B may, for example, point to an improvement in the subject's condition.

In an embodiment, the one or more processors 14 is or are configured to receive a baseline usage of the rescue inhaler by the subject. Such a baseline usage can be provided to the one or more processors 14 in any suitable manner. For example, the baseline usage may be derived, e.g. by the one or more processors 14, from non-exacerbation rescue inhaler uses determined by the use determination system 12B, as previously described.

The one or more processors 14 is or are also configured to receive a post-exacerbation usage of the rescue inhaler as determined via the use determination system 12B.

In some embodiments, the one or more processors 14 is or are further configured to determine, based on a comparison between the post-exacerbation usage and the baseline usage, fulfilment of a rescue inhaler usage criterion for changing the dose of an oral corticosteroid being administered to the subject post-exacerbation from an initial dose.

Alternatively or additionally, the one or more processors 14 is or are configured to receive a baseline value of an inhalation parameter indicative of a baseline lung function of the subject. Such a baseline value can be provided to the one or more processors 14 in any suitable manner. For example, the baseline value may be derived, e.g. by the one or more processors 14, from one or more non-exacerbation values measured by the sensor system 12A, as previously described.

The one or more processors 14 is or are also configured to receive a post-exacerbation value of the inhalation parameter determined from one or more post-exacerbation inhalations using the inhaler(s) 100. Such a post-exacerbation value may be received by the one or more processors 14 from the sensor system 12A.

In some embodiments, the one or more processors 14 is or are further configured to determine, based on a comparison between the post-exacerbation value and the baseline value, fulfilment of an inhalation parameter criterion for changing the dose of an oral corticosteroid being administered to the subject post-exacerbation from an initial dose.

The system 10 may thus provide automated determination of when the inhalation parameter criterion and/or the rescue inhaler usage criterion is or are fulfilled such that the dose of the oral corticosteroid can be changed from the initial dose.

Whilst not visible in FIG. 1, the system 10 may include a user interface configured to provide an output responsive to the determination of fulfilment of the inhalation parameter criterion and/or the rescue inhaler usage criterion. Such an output may, for instance, be in the form of a notification to a healthcare provider. Such a notification may, for example, include an indication of if, when and/or how the dose of the oral corticosteroid should be changed from the initial dose, as previously described.

Alternatively or additionally, the system 10 may comprise a medication container, such as a pill box (not visible), in communication, e.g. wireless communication, with the one or more processors 14. The medication container contains the oral corticosteroid, e.g. in tablet or delayed release tablet form. In such a non-limiting example, the medication container may be responsive to the determination that the inhalation parameter criterion and/or the rescue inhaler usage criterion is or are fulfilled such as to assist the subject to change the dose of the oral corticosteroid from the initial dose.

This can be implemented in any suitable manner. The medication container may include a user interface which is controlled by the one or more processors to provide an indication concerning if, when and/or how the dose of the oral corticosteroid should be changed from the initial dose. Alternatively or additionally, the medication container may comprise a lockable component configured to restrict access to at least some dosage forms of the oral corticosteroid in the medication container in response to the determination that the inhalation parameter criterion and/or the rescue inhaler usage criterion is or are fulfilled. This may assist the patient to change from the initial dose of the oral corticosteroid to a different, for example lower, dose in accordance with fulfilment of the inhalation parameter criterion and/or the rescue inhaler usage criterion.

In another embodiment, the one or more processors 14 is or are configured to receive a baseline value of an inhalation parameter indicative of a baseline lung function of a subject, and receive a post-exacerbation value of the inhalation parameter determined from one or more post-exacerbation inhalations performed by the subject using the inhaler 100. Such a post-exacerbation value may be received by the one or more processors 14 from the sensor system 12A, as previously described. The one or more processors 14 is or are further configured to control a user interface, e.g. comprising or in the form of a display device (not visible in FIG. 1), to communicate the baseline value, and control the user interface to communicate the post-exacerbation value such as to permit comparison between the post-exacerbation value and the baseline value.

Alternatively or additionally, the one or more processors 14 is or are configured to receive a baseline usage of the rescue inhaler by the subject. The one or more processors 14 is or are also configured to receive a post-exacerbation usage of the rescue inhaler. Such a post-exacerbation usage may be received by the one or more processors 14 from the use determination system 12B, as previously described. The one or more processors 14 is or are further configured to control a user interface, e.g. comprising or in the form of a display device (not visible in FIG. 1), to communicate the baseline usage of the rescue inhaler, and control the user interface to communicate the post-exacerbation usage of the rescue inhaler such as to permit comparison between the post-exacerbation usage and the baseline usage.

In this way, the system 10 assists the user, e.g. a clinician, in the technical task of determining whether and/or how to change the dose of the oral corticosteroid from the initial dose.

More generally, the one or more processors 14 of the system 10 may be provided and implemented in any suitable manner. In a non-limiting example, the one or more processors 14 may be provided separately from the respective first and/or second inhaler(s), in which case the one or more processors 14 receive(s) the number of rescue inhalations and parameter data transmitted thereto from the sensor system 12A and the use determination system 12B of the first and/or second inhalers. By processing the data in such an external processing unit, such as in the processing unit of an external device, or in a server, e.g. a remote server, the battery life of the inhaler may be advantageously preserved.

In an alternative non-limiting example, the one or more processors 14 may be an integral part of the first and/or second inhaler, for example contained within a main housing or top cap (not shown in FIG. 1) of the first and/or second inhaler. In such an example, connectivity to an external device need not be relied upon.

It may also be contemplated that some of the functions of the one or more processors 14 may be performed by an internal processing unit included in the first and/or second inhaler and other functions of the one or more processors 14 may be performed by the external processing unit.

More generally, the system 10 may include, for example, a communication module (not shown in FIG. 1) configured to communicate the determined post-exacerbation rescue inhaler usage and/or post-exacerbation value of the inhalation parameter to the subject and/or a healthcare provider, such as a clinician. The subject and/or the clinician may then take appropriate steps accordingly. When, for instance, a smart phone processing unit is included in the processor, the communication functions of the smart phone, such as SMS, email, Bluetooth®, etc., may be employed to communicate the determined the determined post-exacerbation rescue inhaler usage and/or post-exacerbation value to the healthcare provider.

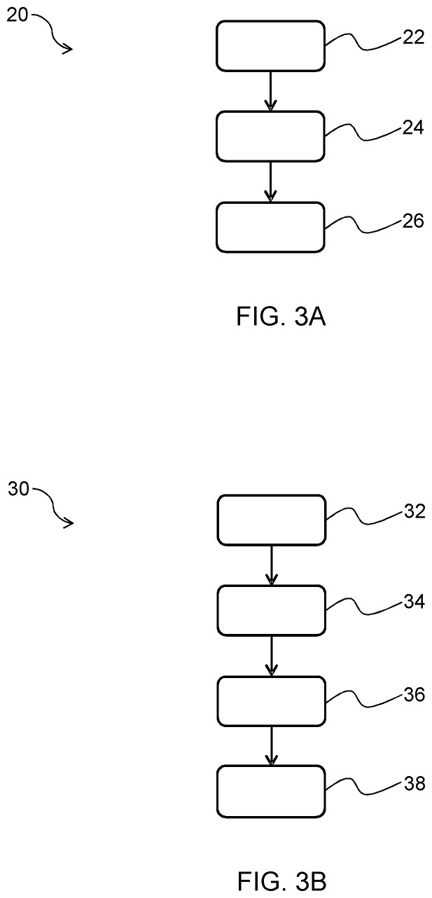

FIG. 2 shows a non-limiting example of a system 10. The system 10 includes the first inhaler 100, an external device 15 (e.g. a mobile device), a public and/or private network 16 (e.g. the internet, a cloud network, etc.), and a personal data storage device 17. The external device 15 may, for example, include a smart phone, a personal computer, a laptop, a wireless-capable media device, a media streaming device, a tablet device, a wearable device, a Wi-Fi or wireless-communication-capable television, or any other suitable internet protocol-enabled device. For example, the external device 15 may be configured to transmit and/or receive RF signals via a Wi-Fi communication link, a Wi-MAX communications link, a Bluetooth® or Bluetooth® Smart communications link, a near field communication (NFC) link, a cellular communications link, a television white space (TVWS) communication link, or any combination thereof. The external device 15 may transfer data through the public and/or private network 16 to the personal data storage device 17.

The first inhaler 100 may include a communication circuit, such as a Bluetooth® radio, for transferring data to the external device 15. The data may include the abovementioned post-exacerbation value of the inhalation parameter data and/or post-exacerbation rescue inhaler usage data.

The first inhaler 100 may also, for example, receive data from the external device 15, such as, for example, program instructions, operating system changes, dosage information, alerts or notifications, acknowledgments, etc.

The external device 15 may include at least part of the one or more processors 14, and thereby process, analyze and/or communicate inhalation parameter data. For example, the external device 15 may process the data such as to determine fulfilment of the inhalation parameter criterion and/or rescue inhaler usage criterion, as represented by block 18A, and provide such information to the personal data storage device 17 for remote storage thereon.

In some non-limiting examples, the external device 15 may also process the data to identify no-inhalation events, low inhalation events, good inhalation events, excessive inhalation events and/or exhalation events, as represented by block 18B. The external device 15 may also process the data to identify underuse events, overuse events and optimal use events, as represented by block 18C. The external device 15 may, for instance, process the data to estimate the number of doses delivered and/or remaining and to identify error conditions, such as those associated with a timestamp error flag indicative of failure of the subject to inhale a dose of the medicament which has been metered by the dose metering assembly. The external device 15 may include a display and software for visually presenting the usage parameters through a graphical user interface.

Although illustrated as being stored on the personal data storage device 17, in some examples, at least some of the determinations, as represented by block 18A, the no inhalation events, low inhalations events, good inhalation events, excessive inhalation events and/or exhalation events, as represented by block 18B, and/or the underuse events, overuse events and optimal use events, as represented by block 18C, may be stored on the external device 15.

When the above-described medication container is also included in the system 10, the medication container may include a further communication circuit, such as a Bluetooth® radio, for communicating with the external device 15, and in particular with the one or more processors 14, as previously described.

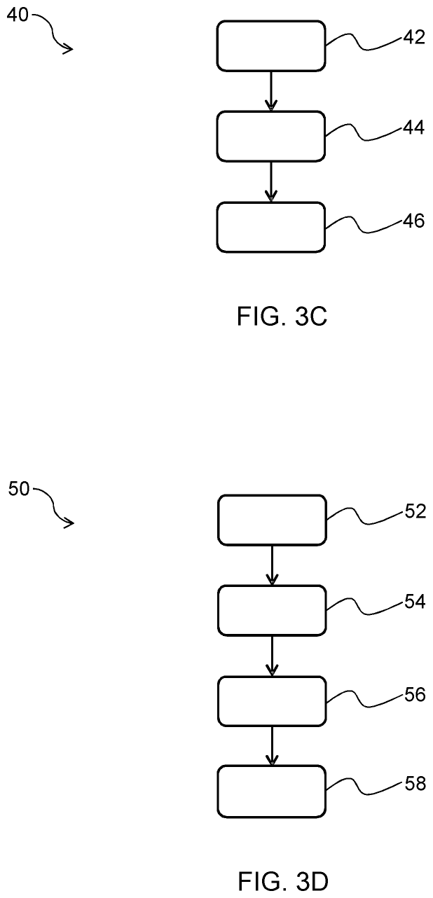

FIGS. 3A to 3D show flowcharts of methods 20, 30, 40, 50 according to embodiments of the present disclosure. The methods 20, 30, 40, 50 may be performed by a system, such as the system 10 illustrated in FIGS. 1 and/or 2. For example, one or more of the first and/or second inhaler, the external device 15, and/or the personal data storage device 17 may be configured to perform the entirety of or a portion of the methods 20, 30, 40, 50. That is, any combination of the steps 22, 24 and 26, 32, 34, 36 and 38, 42, 44 and 46, and 52, 54, 56 and 58 may be performed by any combination of the first inhaler, the second inhaler, the external device 15, and/or the personal data storage device 17.

Turning to FIG. 3A, the method 20 comprises receiving 22 a baseline value of an inhalation parameter indicative of a baseline lung function of the subject, and receiving 24 a post-exacerbation value of the inhalation parameter determined from one or more post-exacerbation inhalations using at least one inhaler. Whilst FIG. 3A shows the receiving 22 the baseline value taking place prior to the receiving 24 the post-exacerbation value, the order of these operations may be reversed.

The method 20 further comprises determining 26, based on a comparison between the post-exacerbation value and the baseline value, fulfilment of an inhalation parameter criterion for, e.g. suitable for, changing the dose of an oral corticosteroid being administered to the subject post-exacerbation from an initial dose.

The method 20 may thus provide automated determination of when the inhalation parameter criterion is fulfilled such that the dose of the oral corticosteroid can be changed from the initial dose.

Turning to FIG. 3B, the method 30 comprises receiving 32 a baseline value of an inhalation parameter indicative of a baseline lung function of a subject, and receiving 34 a post-exacerbation value of the inhalation parameter determined from one or more post-exacerbation inhalations performed by the subject using at least one inhaler.

The method 30 further comprises controlling 36 a user interface to communicate the baseline value, and controlling 38 the user interface to communicate the post-exacerbation value such as to permit comparison between the post-exacerbation value and the at least one baseline value.

It is noted that the order of the steps 32, 34, 36 and 38 depicted in FIG. 3B should not be regarded as being limiting, and the method 30 can be implemented in any suitable order.

Turning to FIG. 3C, the method 40 comprises: receiving 42 a baseline rescue inhaler usage, receiving 44 a post-exacerbation rescue inhaler usage; and determining 46, based on a comparison between the post-exacerbation rescue inhaler usage and the baseline rescue inhaler usage, fulfilment of a rescue inhaler usage criterion for changing the dose of an oral corticosteroid being administered to the subject post-exacerbation from an initial dose.

The method 40 may thus provide automated determination of when the rescue inhaler usage criterion is fulfilled such that the dose of the oral corticosteroid can be changed from the initial dose.

Whilst FIG. 3C shows the receiving 42 the baseline rescue inhaler usage taking place prior to the receiving 44 the post-exacerbation rescue inhaler usage, the order of these operations may be reversed.

Turning to FIG. 3D, the method 50 comprises: receiving 52 a baseline rescue inhaler usage; receiving 54 a post-exacerbation rescue inhaler usage; controlling 56 a user interface to communicate the baseline rescue inhaler usage; and controlling 58 the user interface to communicate the post-exacerbation rescue inhaler usage such as to permit comparison between the post-exacerbation rescue inhaler usage and the baseline rescue inhaler usage.

It is noted that the order of the steps 52, 54, 56 and 58 depicted in FIG. 3D should not be regarded as being limiting, and the method 50 can be implemented in any suitable order.

FIGS. 4-8 provide a non-limiting example of an inhaler which may be included in the system 10.

FIG. 4 provides a front perspective view of a first inhaler 100, according to a non-limiting example. The inhaler 100 may, for example, be a breath-actuated inhaler. The inhaler 100 may include a top cap 102, a main housing 104, a mouthpiece 106, a mouthpiece cover 108, an electronics module 120, and/or an air vent 126. The mouthpiece cover 108 may be hinged to the main housing 104 so that it may open and close to expose the mouthpiece 106. Although illustrated as a hinged connection, the mouthpiece cover 106 may be connected to the inhaler 100 through other types of connections. Moreover, while the electronics module 120 is illustrated as housed within the top cap 102 at the top of the main housing 104, the electronics module 120 may be integrated and/or housed within main body 104 of the inhaler 100.

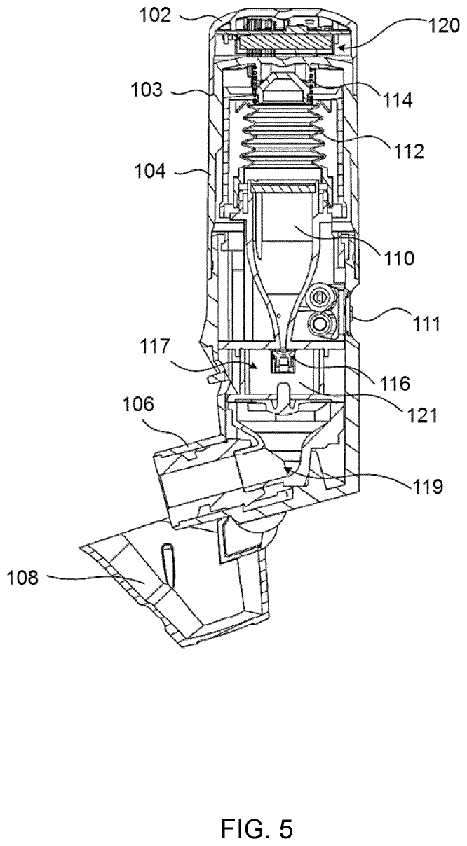

FIG. 5 provides a cross-sectional interior perspective view of the example inhaler 100. Inside the main housing 104, the inhaler 100 may include a medication reservoir 110 (e.g. a hopper), a bellows 112, a bellows spring 114, a yoke (not visible), a dosing cup 116, a dosing chamber 117, a deagglomerator 121, and a flow pathway 119. The medication reservoir 110 may include medication, such as dry powder medication, for delivery to the subject. When the mouthpiece cover 108 is moved from the closed to the open position, the bellows 112 may compress to deliver a dose of medication from the medication reservoir 110 to the dosing cup 116. Thereafter, a subject may inhale through the mouthpiece 106 in an effort to receive the dose of medication.

The airflow generated from the subject's inhalation may cause the deagglomerator 121 to aerosolize the dose of medication by breaking down the agglomerates of the medicament in the dose cup 116. The deagglomerator 121 may be configured to aerosolize the medication when the airflow through the flow pathway 119 meets or exceeds a particular rate, or is within a specific range. When aerosolized, the dose of medication may travel from the dosing cup 116, into the dosing chamber 117, through the flow pathway 119, and out of the mouthpiece 106 to the subject. If the airflow through the flow pathway 119 does not meet or exceed a particular rate, or is not within a specific range, the medication may remain in the dosing cup 116. In the event that the medication in the dosing cup 116 has not been aerosolized by the deagglomerator 121, another dose of medication may not be delivered from the medication reservoir 110 when the mouthpiece cover 108 is subsequently opened. Thus, a single dose of medication may remain in the dosing cup until the dose has been aerosolized by the deagglomerator 121. When a dose of medication is delivered, a dose confirmation may be stored in memory at the inhaler 100 as dose confirmation information.

As the subject inhales through the mouthpiece 106, air may enter the air vent to provide a flow of air for delivery of the medication to the subject. The flow pathway 119 may extend from the dosing chamber 117 to the end of the mouthpiece 106, and include the dosing chamber 117 and the internal portions of the mouthpiece 106. The dosing cup 116 may reside within or adjacent to the dosing chamber 117. Further, the inhaler 100 may include a dose counter 111 that is configured to be initially set to a number of total doses of medication within the medication reservoir 110 and to decrease by one each time the mouthpiece cover 108 is moved from the closed position to the open position.

The top cap 102 may be attached to the main housing 104. For example, the top cap 102 may be attached to the main housing 104 through the use of one or more clips that engage recesses on the main housing 104. The top cap 102 may overlap a portion of the main housing 104 when connected, for example, such that a substantially pneumatic seal exists between the top cap 102 and the main housing 104.

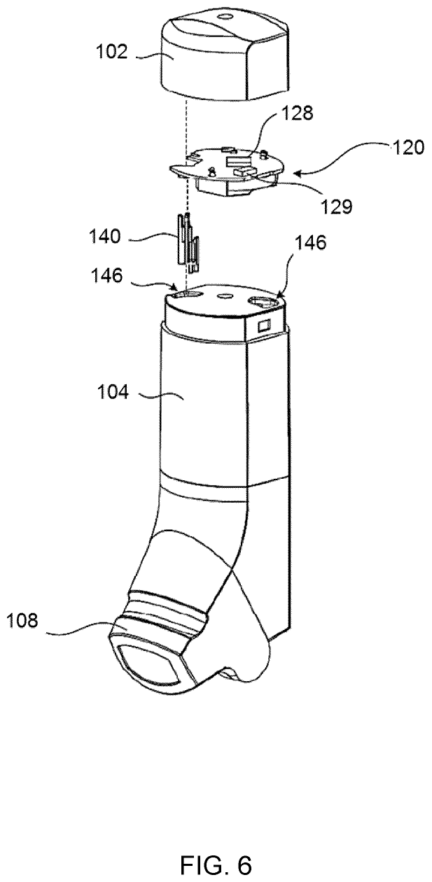

FIG. 6 is an exploded perspective view of the example inhaler 100 with the top cap 102 removed to expose the electronics module 120. As shown in FIG. 6, the top surface of the main housing 104 may include one or more (e.g. two) orifices 146. One of the orifices 146 may be configured to accept a slider 140. For example, when the top cap 102 is attached to the main housing 104, the slider 140 may protrude through the top surface of the main housing 104 via one of the orifices 146.

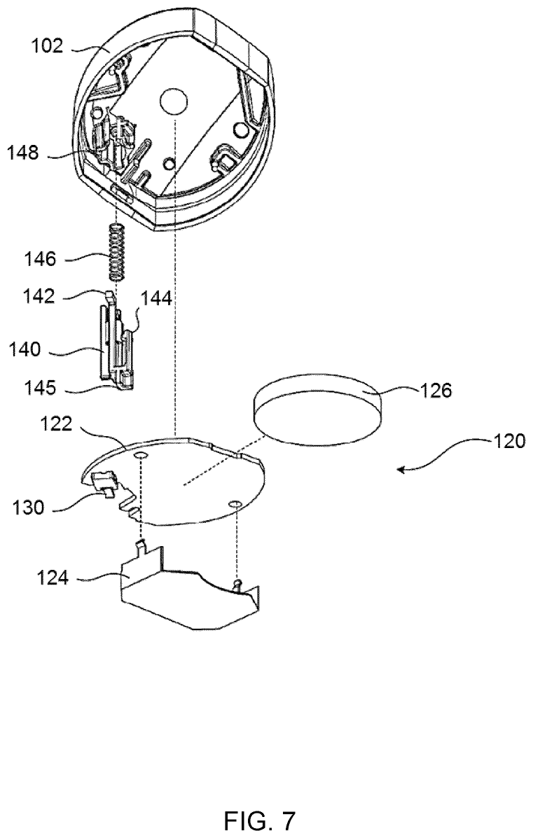

FIG. 7 is an exploded perspective view of the top cap 102 and the electronics module 120 of the example inhaler 100. As shown in FIG. 7, the slider 140 may define an arm 142, a stopper 144, and a distal end 145. The distal end 145 may be a bottom portion of the slider 140. The distal end 145 of the slider 140 may be configured to abut the yoke that resides within the main housing 104 (e.g. when the mouthpiece cover 108 is in the closed or partially open position). The distal end 145 may be configured to abut a top surface of the yoke when the yoke is in any radial orientation. For example, the top surface of the yoke may include a plurality of apertures (not shown), and the distal end 145 of the slider 140 may be configured to abut the top surface of the yoke, for example, whether or not one of the apertures is in alignment with the slider 140.

The top cap 102 may include a slider guide 148 that is configured to receive a slider spring 146 and the slider 140. The slider spring 146 may reside within the slider guide 148. The slider spring 146 may engage an inner surface of the top cap 102, and the slider spring 146 may engage (e.g. abut) an upper portion (e.g. a proximate end) of the slider 140. When the slider 140 is installed within the slider guide 148, the slider spring 146 may be partially compressed between the top of the slider 140 and the inner surface of the top cap 102. For example, the slider spring 146 may be configured such that the distal end 145 of the slider 140 remains in contact with the yoke when the mouthpiece cover 108 is closed. The distal end 145 of the slider 145 may also remain in contact with the yoke while the mouthpiece cover 108 is being opened or closed. The stopper 144 of the slider 140 may engage a stopper of the slider guide 148, for example, such that the slider 140 is retained within the slider guide 148 through the opening and closing of the mouthpiece cover 108, and vice versa. The stopper 144 and the slider guide 148 may be configured to limit the vertical (e.g. axial) travel of the slider 140. This limit may be less than the vertical travel of the yoke. Thus, as the mouthpiece cover 108 is moved to a fully open position, the yoke may continue to move in a vertical direction towards the mouthpiece 106 but the stopper 144 may stop the vertical travel of the slider 140 such that the distal end 145 of the slider 140 may no longer be in contact with the yoke.

More generally, the yoke may be mechanically connected to the mouthpiece cover 108 and configured to move to compress the bellows spring 114 as the mouthpiece cover 108 is opened from the closed position and then release the compressed bellows spring 114 when the mouthpiece cover reaches the fully open position, thereby causing the bellows 112 to deliver the dose from the medication reservoir 110 to the dosing cup 116. The yoke may be in contact with the slider 140 when the mouthpiece cover 108 is in the closed position. The slider 140 may be arranged to be moved by the yoke as the mouthpiece cover 108 is opened from the closed position and separated from the yoke when the mouthpiece cover 108 reaches the fully open position. This arrangement may be regarded as a non-limiting example of the previously described dose metering assembly, since opening the mouthpiece cover 108 causes the metering of the dose of the medicament.

The movement of the slider 140 during the dose metering may cause the slider 140 to engage and actuate a switch 130. The switch 130 may trigger the electronics module 120 to register the dose metering. The slider 140 and switch 130 together with the electronics module 120 may thus correspond to a non-limiting example of the use determination system 12B described above. The slider 140 may be regarded in this example as the means by which the use determination system 12B is configured to register the metering of the dose by the dose metering assembly, each metering being thereby indicative of the inhalation performed by the subject using the inhaler 100.

Actuation of the switch 130 by the slider 140 may also, for example, cause the electronics module 120 to transition from the first power state to a second power state, and to sense an inhalation by the subject from the mouthpiece 106.

The electronics module 120 may include a printed circuit board (PCB) assembly 122, a switch 130, a power supply (e.g. a battery 126), and/or a battery holder 124. The PCB assembly 122 may include surface mounted components, such as a sensor system 128, a wireless communication circuit 129, the switch 130, and or one or more indicators (not shown), such as one or more light emitting diodes (LEDs). The electronics module 120 may include a controller (e.g. a processor) and/or memory. The controller and/or memory may be physically distinct components of the PCB 122. Alternatively, the controller and memory may be part of another chipset mounted on the PCB 122, for example, the wireless communication circuit 129 may include the controller and/or memory for the electronics module 120. The controller of the electronics module 120 may include a microcontroller, a programmable logic device (PLD), a microprocessor, an application specific integrated circuit (ASIC), a field programmable gate array (FPGA), or any suitable processing device or control circuit.

The controller may access information from, and store data in the memory. The memory may include any type of suitable memory, such as non-removable memory and/or removable memory. The non-removable memory may include random-access memory (RAM), read-only memory (ROM), or any other type of memory storage device. The removable memory may include a subscriber identity module (SIM) card, a memory stick, a secure digital (SD) memory card, and the like. The memory may be internal to the controller. The controller may also access data from, and store data in, memory that is not physically located within the electronics module 120, such as on a server or a smart phone.

The sensor system 128 may include one or more sensors. The sensor system 128 may be an example of the sensor system 12A. The sensor system 128 may include one or more sensors, for example, of different types, such as, but not limited to one or more pressure sensors, temperature sensors, humidity sensors, orientation sensors, acoustic sensors, and/or optical sensors. The one or more pressure sensors may include a barometric pressure sensor (e.g. an atmospheric pressure sensor), a differential pressure sensor, an absolute pressure sensor, and/or the like. The sensors may employ microelectromechanical systems (MEMS) and/or nanoelectromechanical systems (NEMS) technology.

The sensor system 128 may be configured to provide an instantaneous reading (e.g. pressure reading) to the controller of the electronics module 120 and/or aggregated readings (e.g. pressure readings) over time. As illustrated in FIGS. 5 and 6, the sensor system 128 may reside outside the flow pathway 119 of the inhaler 100, but may be pneumatically coupled to the flow pathway 119.

The controller of the electronics module 120 may receive signals corresponding to measurements from the sensor system 128. The controller may calculate or determine one or more airflow metrics using the signals received from the sensor system 128. The airflow metrics may be indicative of a profile of airflow through the flow pathway 119 of the inhaler 100. For example, if the sensor system 128 records a change in pressure of 0.3 kilopascals (kPa), the electronics module 120 may determine that the change corresponds to an airflow rate of approximately 45 liters per minute (Lpm) through the flow pathway 119.

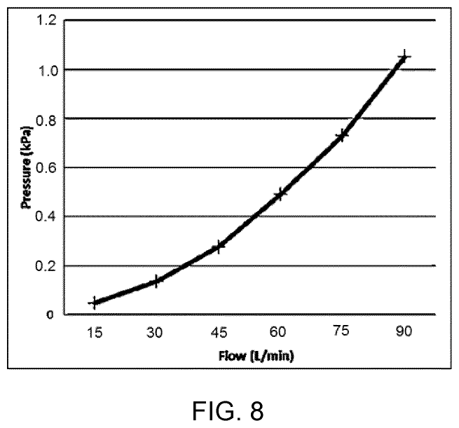

FIG. 8 shows a graph of airflow rates versus pressure. The airflow rates and profile shown in FIG. 8 are merely examples and the determined rates may depend on the size, shape, and design of the inhalation deice 100 and its components.

The one or more processors 14 may generate personalized data in real-time by comparing signals received from the sensor system 128 and/or the determined airflow metrics to one or more thresholds or ranges, for example, as part of an assessment of how the inhaler 100 is being used and/or whether the use is likely to result in the delivery of a full dose of medication. For example, where the determined airflow metric corresponds to an inhalation with an airflow rate below a particular threshold, the one or more processors 14 may determine that there has been no inhalation or an insufficient inhalation from the mouthpiece 106 of the inhaler 100. If the determined airflow metric corresponds to an inhalation with an airflow rate above a particular threshold, the one or more processors 14 may determine that there has been an excessive inhalation from the mouthpiece 106. If the determined airflow metric corresponds to an inhalation with an airflow rate within a particular range, the one or more processors 14 may determine that the inhalation is “good”, or likely to result in a full dose of medication being delivered.

The pressure measurement readings and/or the computed airflow metrics may be indicative of the quality or strength of inhalation from the inhaler 100. For example, when compared to a particular threshold or range of values, the readings and/or metrics may be used to categorize the inhalation as a certain type of event, such as a good inhalation event, a low inhalation event, a no inhalation event, or an excessive inhalation event. The categorization of the inhalation may be usage parameters stored as personalized data of the subject.

The no inhalation event may be associated with pressure measurement readings and/or airflow metrics below a particular threshold, such as an airflow rate less than 30 Lpm. The no inhalation event may occur when a subject does not inhale from the mouthpiece 106 after opening the mouthpiece cover 108 and during the measurement cycle. The no inhalation event may also occur when the subject's inspiratory effort is insufficient to ensure proper delivery of the medication via the flow pathway 119, such as when the inspiratory effort generates insufficient airflow to activate the deagglomerator 121 and, thus, aerosolize the medication in the dosing cup 116.

The low inhalation event may be associated with pressure measurement readings and/or airflow metrics within a particular range, such as an airflow rate between 30 Lpm and 45 Lpm. The low inhalation event may occur when the subject inhales from the mouthpiece 106 after opening the mouthpiece cover 108 and the subject's inspiratory effort causes at least a partial dose of the medication to be delivered via the flow pathway 119. That is, the inhalation may be sufficient to activate the deagglomerator 121 such that at least a portion of the medication is aerosolized from the dosing cup 116.

The good inhalation event may be associated with pressure measurement readings and/or airflow metrics above the low inhalation event, such as an airflow rate between 45 Lpm and 200 Lpm. The good inhalation event may occur when the subject inhales from the mouthpiece 106 after opening the mouthpiece cover 108 and the subject's inspiratory effort is sufficient to ensure proper delivery of the medication via the flow pathway 119, such as when the inspiratory effort generates sufficient airflow to activate the deagglomerator 121 and aerosolize a full dose of medication in the dosing cup 116.

The excessive inhalation event may be associated with pressure measurement readings and/or airflow metrics above the good inhalation event, such as an airflow rate above 200 Lpm. The excessive inhalation event may occur when the subject's inspiratory effort exceeds the normal operational parameters of the inhaler 100. The excessive inhalation event may also occur if the device 100 is not properly positioned or held during use, even if the subject's inspiratory effort is within a normal range. For example, the computed airflow rate may exceed 200 Lpm if the air vent is blocked or obstructed (e.g. by a finger or thumb) while the subject is inhaling from the mouthpiece 106.

Any suitable thresholds or ranges may be used to categorize a particular event. Some or all of the events may be used. For example, the no inhalation event may be associated with an airflow rate below 45 Lpm and the good inhalation event may be associated with an airflow rate between 45 Lpm and 200 Lpm. As such, the low inhalation event may not be used at all in some cases.

The pressure measurement readings and/or the computed airflow metrics may also be indicative of the direction of flow through the flow pathway 119 of the inhaler 100. For example, if the pressure measurement readings reflect a negative change in pressure, the readings may be indicative of air flowing out of the mouthpiece 106 via the flow pathway 119. If the pressure measurement readings reflect a positive change in pressure, the readings may be indicative of air flowing into the mouthpiece 106 via the flow pathway 119. Accordingly, the pressure measurement readings and/or airflow metrics may be used to determine whether a subject is exhaling into the mouthpiece 106, which may signal that the subject is not using the device 100 properly.

The personalized data collected from, or calculated based on, the usage of the inhaler 100 (e.g. pressure metrics, airflow metrics, lung function metrics, dose confirmation information, etc.) may be computed and/or assessed via external devices as well (e.g. partially or entirely). More specifically, the wireless communication circuit 129 in the electronics module 120 may include a transmitter and/or receiver (e.g. a transceiver), as well as additional circuitry. For example, the wireless communication circuit 129 may include a Bluetooth chip set (e.g. a Bluetooth Low Energy chip set), a ZigBee chipset, a Thread chipset, etc. As such, the electronics module 120 may wirelessly provide the personalized data, such as pressure measurements, airflow metrics, lung function metrics, dose confirmation information, and/or other conditions related to usage of the inhaler 100, to an external device, including a smart phone. The personalized data may be provided in real time to the external device to enable the above-described probability determination based on real-time data from the inhaler 100 that indicates time of use, how the inhaler 100 is being used, and personalized data about the user of the inhaler, such as real-time data related to the subject's lung function and/or medical treatment. The external device may include software for processing the received information and for providing compliance and adherence feedback to users of the inhaler 100 via a graphical user interface (GUI).

The airflow metrics may include personalized data that is collected from the inhaler 100 in real-time, such as one or more of an average flow of an inhalation/exhalation, a peak flow of an inhalation/exhalation (e.g. a maximum inhalation received), a volume of an inhalation/exhalation, a time to peak of an inhalation/exhalation, and/or the duration of an inhalation/exhalation. The airflow metrics may also be indicative of the direction of flow through the flow pathway 119. That is, a negative change in pressure may correspond to an inhalation from the mouthpiece 106, while a positive change in pressure may correspond to an exhalation into the mouthpiece 106. When calculating the airflow metrics, the electronics module 120 may be configured to eliminate or minimize any distortions caused by environmental conditions. For example, the electronics module 120 may re-zero to account for changes in atmospheric pressure before or after calculating the airflow metrics. The one or more pressure measurements and/or airflow metrics may be timestamped and stored in the memory of the electronics module 120.

In addition to the airflow metrics, the inhaler 100, or another computing device, may use the airflow metrics to generate additional personalized data. For example, the controller of the electronics module 120 of the inhaler 100 may translate the airflow metrics into other metrics that indicate the subject's lung function and/or lung health that are understood to medical practitioners, such as peak inspiratory flow metrics, peak expiratory flow metrics, and/or forced expiratory volume in 1 second (FEV1), for example. The electronics module 120 of the inhaler may determine a measure of the subject's lung function and/or lung health using a mathematical model such as a regression model. The mathematical model may identify a correlation between the total volume of an inhalation and FEV1. The mathematical model may identify a correlation between peak inspiratory flow and FEV1. The mathematical model may identify a correlation between the total volume of an inhalation and peak expiratory flow. The mathematical model may identify a correlation between peak inspiratory flow and peak expiratory flow.

The battery 126 may provide power to the components of the PCB 122. The battery 126 may be any suitable source for powering the electronics module 120, such as a coin cell battery, for example. The battery 126 may be rechargeable or non-rechargeable. The battery 126 may be housed by the battery holder 124. The battery holder 124 may be secured to the PCB 122 such that the battery 126 maintains continuous contact with the PCB 122 and/or is in electrical connection with the components of the PCB 122. The battery 126 may have a particular battery capacity that may affect the life of the battery 126. As will be further discussed below, the distribution of power from the battery 126 to the one or more components of the PCB 122 may be managed to ensure the battery 126 can power the electronics module 120 over the useful life of the inhaler 100 and/or the medication contained therein.

In a connected state, the communication circuit and memory may be powered on and the electronics module 120 may be “paired” with an external device, such as a smart phone. The controller may retrieve data from the memory and wirelessly transmit the data to the external device. The controller may retrieve and transmit the data currently stored in the memory. The controller may also retrieve and transmit a portion of the data currently stored in the memory. For example, the controller may be able to determine which portions have already been transmitted to the external device and then transmit the portion(s) that have not been previously transmitted. Alternatively, the external device may request specific data from the controller, such as any data that has been collected by the electronics module 120 after a particular time or after the last transmission to the external device. The controller may retrieve the specific data, if any, from the memory and transmit the specific data to the external device.

The data stored in the memory of the electronics module 120 (e.g. the signals generated by the switch 130, the pressure measurement readings taken by the sensory system 128 and/or the airflow metrics computed by the controller of the PCB 122) may be transmitted to an external device, which may process and analyze the data to determine the usage parameters associated with the inhaler 100. Further, a mobile application residing on the mobile device may generate feedback for the user based on data received from the electronics module 120. For example, the mobile application may generate daily, weekly, or monthly report, provide confirmation of error events or notifications, provide instructive feedback to the subject, and/or the like.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

What is claimed is:

1. (canceled)

2. The method of claim 1, wherein said at least one inhaler comprises at least one of:
a maintenance inhaler configured to deliver a maintenance medicament to the subject during the one or more post-exacerbation inhalations; or
a rescue inhaler configured to deliver a rescue medicament to the subject during the one or more post-exacerbation inhalations.

3. (canceled)

4. The method of claim 1, wherein the dose of the oral corticosteroid is lowered relative to the initial dose responsive to fulfilment of said inhalation parameter criterion or fulfilment of said rescue inhaler usage criterion.

5. (canceled)

6. The method of claim 1, wherein the subject has a baseline rescue inhaler usage, and wherein fulfilment of the rescue inhaler usage criterion is determined based on a comparison of a post-exacerbation rescue inhaler usage and the baseline rescue inhaler usage.

7. The pharmaceutical composition for the use according to any of claims 1 to 6, wherein the subject has a baseline lung function indicated by a baseline value of an inhalation parameter, and wherein fulfilment of the inhalation parameter criterion is determined based on a comparison of a post-exacerbation value of the inhalation parameter obtained from said one or more post-exacerbation inhalations to said baseline value.

8. The method of claim 7, wherein the inhalation parameter criterion is fulfilled by said post-exacerbation value being within a predetermined tolerance defined for the baseline value.

9. The method of claim 7, wherein the inhalation parameter comprises any one or a combination of two or more of:
a peak inhalation flow;
an inhalation volume;
an inhalation duration; and
a time taken to reach peak inhalation flow.

10.-12. (canceled)

13. The method claim 7, wherein the post-exacerbation value of the inhalation parameter increases towards the baseline value during reversion to said baseline lung function, and wherein the inhalation parameter criterion is fulfilled by said post-exacerbation value being at least 70% of the baseline value.

14. (canceled)

15. (canceled)

16. The method of claim 7, wherein the post-exacerbation value comprises an average of a plurality of post-exacerbation values of the inhalation parameter obtained from a plurality of said post-exacerbation inhalations.

17. (canceled)

18. The method of claim 1, wherein the pharmaceutical composition is in tablet form.

19. (canceled)

20. (canceled)

21. The method of claim 1, wherein the oral corticosteroid comprises prednisone or prednisolone.

22. The method of pharmaceutical composition for the use according to claim 21, wherein said initial dose is in the range of 5 to 60 mg daily for adults aged 18 years and older.

23.-25. (canceled)

26. The method of claim 21, wherein said initial dose is in the range of 40 to 50 mg daily for children aged 12 to 17 years.

27. The method of claim 21, wherein said initial dose is in the range of 1 to 2 mg/kg daily for children aged 1 month to 11 years.

28. The method of claim 1, wherein the respiratory disease is chronic obstructive pulmonary disease or asthma.

29. (canceled)

30. A method of post-exacerbation treatment of a respiratory disease in a subject, the method comprising:
treating the subject with an initial dose of an oral corticosteroid until fulfilment of an inhalation parameter criterion by one or more post-exacerbation inhalations performed by the subject with at least one inhaler or fulfilment of a rescue inhaler usage criterion relating to post-exacerbation usage of a rescue inhaler configured to deliver a rescue medicament to the subject, and
responsible to said fulfilment, changing the dose of the oral corticosteroid from the initial dose.

31. (canceled)

32. (canceled)

33. A method comprising:
receiving a baseline value of an inhalation parameter indicative of a baseline lung function of a subject;
receiving a post-exacerbation value of the inhalation parameter determined from one or more post-exacerbation inhalations performed by the subject using at least one inhaler;
controlling a user interface to communicate the baseline value; and
controlling the user interface to communicate the post-exacerbation value such as to permit comparison between the post-exacerbation value and the baseline value.

34. A method comprising:
receiving a baseline rescue inhaler usage;
receiving a post-exacerbation rescue inhaler usage;
controlling a user interface to communicate the baseline rescue inhaler usage; and
controlling the user interface to communicate the post-exacerbation rescue inhaler usage such as to permit comparison between the post-exacerbation rescue inhaler usage and the baseline rescue inhaler usage.

35. The method of claim 33, wherein said baseline value is determined from one or more inhalations using the at least one inhaler when the subject is not experiencing an exacerbation.

36. The method of claim 34, wherein said baseline rescue inhaler usage is determined when the subject is not experiencing an exacerbation.

37. (canceled)

38. One or more non-transitory computer readable media having a computer program stored thereon, the computer program comprising computer program code which is configured, when said computer program is run on one or more physical computing devices, to cause said one or more physical computing devices to implement the method of claim 31.

39.-44.
